(19) United States
(12) Patent Application Publication
(10) Pub. No.: US 2003/0016833 A1
(43) Pub. Date: Jan. 23, 2003

(54) Active noise cancellation system utilizing a signal delay to accommodate noise phase change

(75) Inventor: Bruce James Harvey, Shelby Township, MI (US)

Correspondence Address: JEFFREY P. MORRIS, SIEMENS CORPORATION, 186 WOOD AVENUE SOUTH, ISELIN, NJ, 08830, US

(73) Assignee: Siemens VDO Automotive, Inc.

(21) Appl. No.: 10/195,735

(22) Filed: Jul. 15, 2002

Related U.S. Application Data
(60) Provisional application No. 60/306,551, filed on Jul. 19, 2001.

(51) Int. Cl. A61F 11/06; G10K 11/16; H03B 29/00

(52) U.S. Cl. 381/71.5

(57) ABSTRACT

An active noise cancellation system (20) for use with an assembly (22) includes adjusting the phase of a reference signal an amount that corresponds to a change in phase of the noise (26) within the system. A non-acoustic sensor (44) provides a signal regarding a position or frequency of a source of noise (28) (i.e., RPM's of a vehicle engine). Control electronics (32) provide a reference signal to the system noise canceling actuator (30) (i.e., a speaker) that includes the phase adjustment responsive to the sensor signal. In one example, the reference signal is delayed an amount of time corresponding to the phase change in the noise (26) as it travels through the system (22). In another example, the phase angle is adjusted.

### CROSS REFERENCE TO RELATED APPLICATIONS

This application claims priority to U.S. Provisional Application No. 60/306,551, which was filed on Jul. 19, 2001.

### BACKGROUND OF THE INVENTION

This invention generally relates to noise cancellation. More particularly, this invention relates to controlling signal timing in a noise cancellation system.

Noise cancellation systems have a variety of uses. One example use is on automotive vehicles for reducing noise propagation into the passenger compartment.

Modem day vehicles typically include an air induction system. One drawback of air induction systems is that engine noise frequently travels through the air induction system and emanates out of the mouth of the air intake such that the noises are noticeable in the passenger compartment. This is particularly true under wide open throttle conditions. Various efforts have been made to reduce the amount of engine noise traveling through the air induction system. Some arrangements include using passive devices such as expansion chambers and Helmholtz resonators. Other efforts include active methods such as anti-noise generators.

Other sources of noise may be associated with the vehicle exhaust or a supercharger, for example. Regardless of the particular application, various challenges exist when designing an effective and economical noise cancellation system.

Typical active systems include a speaker that generates a sound to attenuate the noise. The sound from the speaker typically is out of phase with the noise and combines with the noise such that the result is a reduced noise, which results in less noise transmission into the passenger compartment, for example. The speaker sound can be referred to as a noise cancellation signal.

Digital signal processors such as microprocessors typically generate cancellation signals. The microprocessor typically requires some input from the relevant environment to adequately address the need for noise cancellation. In some examples, computer modeling is used so that the microprocessor is able to provide a desired level of noise cancellation. One example aspect of the modeling is to compensate for delays between the speaker and a microphone that provides feedback information to the controller regarding the active noise cancellation system performance.

While such systems are useful, they do not address all difficulties associated with operating an effective noise cancellation system. For example, it is often difficult to adequately track the propagation of noise so that the proper phase relationship between the cancellation signal and the noise results in the desired amount of noise cancellation. One attempt at correcting this problem includes providing a feed forward sensor that provides an indication of the position or frequency of the source of noise (i.e., the vehicle engine). Even with such sensors, however, it is still necessary to utilize relatively complex filtering techniques to attempt to adequately cancel out the engine noise.

This invention provides an enhancement to active noise cancellation that reduces the burden on filters within the system and provides a more accurate and reliable method of canceling engine noise in a vehicle air intake assembly.

### SUMMARY OF THE INVENTION

In general terms, this invention is a system for canceling noise that includes adjusting the phase of a reference signal so that more accurate noise cancellation is achievable.

A system designed according to this invention includes a non-acoustic sensor that provides a signal related to the position or frequency of a source of noise (i.e., a vehicle engine). An actuator is positioned some distance from the source of noise. Control electronics provide a noise canceling reference signal to the actuator so that an appropriate noise cancellation signal is provided by the actuator. The control electronics adjust the phase of the reference signal responsive to the sensor signal in a manner corresponding to a phase change in the noise that occurs as the noise travels from the source to the location(s) where the noise can be cancelled out by the actuator.

Accordingly, the inventive system adjusts the phase of the noise cancellation reference signal. In one example, the control electronics introduce a delay in the signal. In another example, the control electronics introduce a shift in the phase angle of the reference signal. The adjustment preferably corresponds to a phase change in the noise as the noise propagates through the corresponding system. The delay or phase shift in the reference signal accommodates the phase change and, therefore, reduces the need for complex filtering so that the actual noise cancellation signal that drives the actuator will track the noise within the system.

A method of controlling a noise cancellation system according to this invention includes determining a phase change in the noise as the noise travels from the source to the location of the actuator of the noise cancellation system. Modifying the reference signal based upon information from a non-acoustic sensor that provides information regarding a position or frequency of the source of noise accommodates the determined phase change.

The various features and advantages of this invention will become apparent to those skilled in the art from the following detailed description of the currently preferred embodiment. The drawings that accompany the detailed description can be briefly described as follows.

### BRIEF DESCRIPTION OF THE DRAWINGS

FIG. 1 schematically illustrates an active noise cancellation system designed according to this invention.

FIG. 2 is a graphical illustration of a relationship between the phase of the noise and the operating condition of the source of noise, at various operating conditions.

### DETAILED DESCRIPTION OF THE PREFERRED EMBODIMENTS

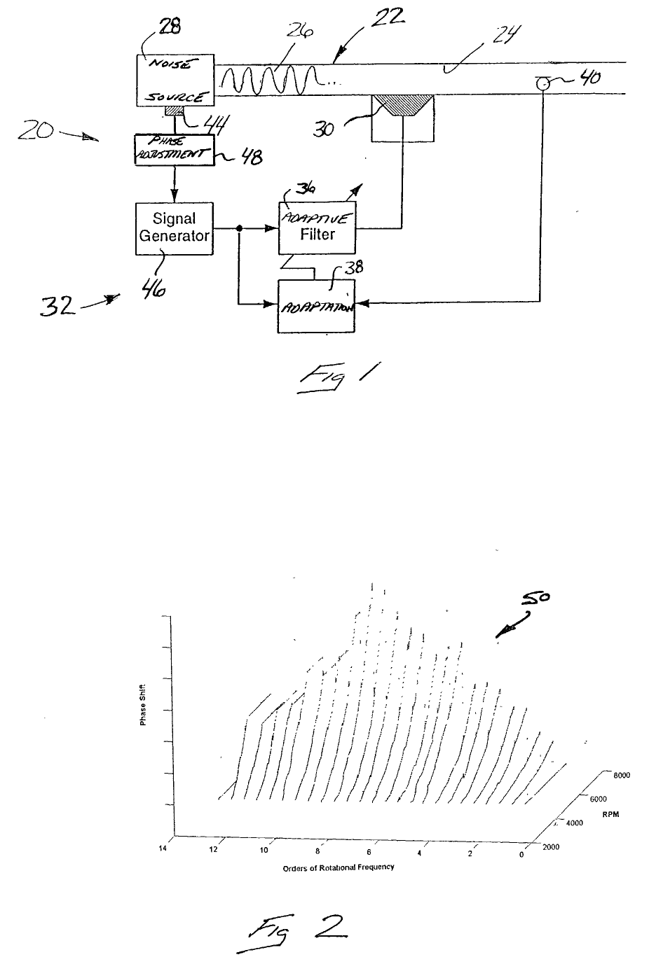

FIG. 1 schematically illustrates an active noise cancellation system 20. An assembly 22 is schematically illustrated. A structure 24 serves a purpose within the assembly 22 but also provides a path for noise to travel along in a manner that makes noise cancellation desirable or necessary. Noise, schematically illustrated at 26, propagates through the structure 24 as caused by operation of the noise source 28. More specifically, the noise 26 is typically related to a position, frequency of the noise source or a change in the operation or some other characteristic of the noise source.

The noise cancellation system 20 includes an actuator 30 that provides a cancellation signal to counteract the noise 26 to eliminate or reduce the amount of noise propagated through the system. Various known actuators, such as a speaker, piezoelectric devices, hydraulic devices or a transducer could be used in a system designed according to this invention but a speaker will be used in this discussion for purposes of illustration. Noise cancellation control electronics 32 drive the speaker 30 to achieve the desired amount of noise cancellation including a signal control strategy that addresses the situations where there is a phase change in the noise 26 as the noise propagates from the noise source 28 through the structure 24.

The control electronics 32 include programming and suitable electronic devices such as a microprocessor or digital signal processor. Such components are well known and those skilled in the art that have the benefit of this description will be able to select from among available components and can then write appropriate software to meet the needs of their particular situation. The schematic divisions in the illustrated control electronics 32 are for discussion purposes only. The various modules and functions may be implemented using a variety of components, software or both.

The signals used to drive the speaker 30 are at least partially determined from feedback information obtained through a microphone 40, which is supported in a known manner. Feedback information from the microphone 40 provides an adaptation portion 38 of the control and an associated adaptive filter 36 with information, for example, regarding the effectiveness of the cancellation signal provided by the speaker 30. Such feedback information allows the control electronics such as the adaptation portion 38 and the filter 36 to alter the signals used to drive the speaker 30 to achieve a more desirable amount of noise cancellation, for example.

The illustrated embodiment includes a non-acoustic sensor 44 associated with the noise source 28 to provide an indication of position or frequency of the noise source 28. In one particular example, the non-acoustic sensor 44 senses the RPM's of a vehicle engine. A signal from the sensor 44 is provided to a signal generator 46 that responsively generates a noise cancellation reference signal so that the timing for generating the cancellation signal by the speaker 30 can achieve the desired amount of noise cancellation.

As the noise 26 propagates from the source 28 to the location of the speaker 30, there is a change in phase in the noise. This phase change, depends at least in part on the position or frequency (i.e., RPM's of an engine) of the noise source 28. The inventive arrangement includes a phase adjustment module 48 that adjusts the phase of the reference signal that is provided by the signal generator 46 in an amount that compensates for the change in phase of the noise. In one example, the phase adjustment comprises a shift in the phase angle of the reference signal. In another example, the adjustment comprises a delay in the noise cancellation reference signal an amount of delay time that corresponds to the phase change of the noise 26. For purposes of discussion, the latter approach will be discussed although either approach is within the scope of this invention.

The desired phase adjustment is determined according to one example by operating the system 20 under a plurality of operating conditions without implementing a time delay in the reference signal. This provides a base line of data regarding the phase change in the noise 26 associated with the various operating conditions. The control electronics 32 then can be programmed to accommodate the noise phase changes by associating the appropriate time delay with the reference signal provided by the signal generator 46.

In one example, the control electronics 32 are programmed to determine a desired time delay in the reference signal based upon information regarding an operation of the noise source 28. Such information can be obtained from a conventional engine controller or commercially available sensors, for example. In another example, a plurality of delay times associated with various operating conditions (i.e., different RPM's) are predetermined and stored within a look up table for use by the phase adjustment module 48 or another appropriate portion of the control electronics 32 that is responsible for introducing the time delay in the reference signal.

This invention includes the ability to provide a plurality of time delays associated with reference signals for canceling noise at a plurality of orders. In one example, a first order is associated with the actual noise source operating condition. A second order is twice the actual operation, a third order is three times the actual operation, etc. This invention accommodates noise cancellation at any selected number of orders and provides the ability to have a different time delay (or phase angle shift) in the reference signal associated with each order.

FIG. 2 graphically illustrates a relationship between the phase of the noise 26 and engine RPM's in a plot 50 for an example where an engine is the noise source at each of a plurality of orders. As can be appreciated from the drawing, significantly different amounts of phase change occur in the different orders. Accordingly, the control electronics 32 preferably accommodate a variety of time delays in the reference signal generation for each of the orders as needed in a particular situation or for a particular vehicle configuration.

In one example, the time delay is determined using the formula: time delay=((change in phase)/(change in RPM))/(6×order number). The factor of 6 is utilized to convert from minutes to seconds and from revolutions to degrees in this example formula. This particular formula is especially useful in a situation where the change in phase can be represented by a straight line.

Because the noise phase change for different orders may exhibit characteristics that are not adequately represented by a straight line, a look up table approach may prove more useful then a programmed calculation approach. Those skilled in the art who have the benefit of this description will be able to decide what approach works best for their particular situation.

Without this invention, the filter 36 would need to track or “chase” the changing phase of the noise at each order of interest. Such a filter introduces additional complexity and expense into a noise cancellation system. The inventive arrangement is more economical, more reliable, more robust and provides more accurate noise cancellation.

The preceding description is exemplary rather than limiting in nature. Variations and modifications to the disclosed examples may become apparent to those skilled in the art that do not necessarily depart from the essence of this invention. The scope of legal protection given to this invention can only be determined by studying the following claims.

I claim:

1. A noise cancellation system, comprising:
a nonacoustic sensor that provides a signal indicating a position or frequency of a source of noise;
an actuator that is adapted to provide a noise cancellation signal and is positioned a distance from the source of noise;
a signal generator that is responsive to the signal from the sensor and provides a noise canceling reference signal to cause the actuator to produce the noise cancellation signal, the signal generator adjusting the phase of the reference signal corresponding to a phase change that occurs as the noise travels from the source to the speaker location.

2. The system of claim 1, including a controller that controls operation of the signal generator and wherein the controller determines a different delay time for each of a plurality of orders and the signal generator delays the reference signal for each order according to the determined delay times.

3. The system of claim 1, wherein the noise source is a vehicle engine and the signal generator determines a delay time based upon a relationship between the phase change and a rate of rotations per minute of the engine.

4. The system of claim 3, wherein the signal generator is programmed to determine the delay time based upon the relationship.

5. The system of claim 3, wherein the signal generator includes a lookup table of a plurality of delay times and wherein the signal generator chooses one of the delay times responsive to information regarding the rate of rotations per minute.

6. The system of claim 1, wherein the signal generator shifts the phase angle of the reference signal.

7. The system of claim 1, including a microphone that provides information regarding the noise and wherein the signal generator delays the reference signal to accommodate signal processing delays associated with receiving information from the microphone and driving the actuator.

8. A method of controlling a noise cancellation system having a nonacoustic sensor that provides a signal indicating a position or frequency of a source of noise, an actuator positioned a set distance from the source of noise and a signal generator that provides a noise canceling reference signal responsive to the signal from the sensor, comprising the steps of:
determining a phase change in the noise as the noise travels from the source to the speaker location; and
adjusting the phase of the reference signal an amount corresponding to the phase change.

9. The method of claim 8, including determining the phase change for each of a plurality of orders and delaying the reference signals a different time for each of the orders.

10. The method of claim 8, wherein the noise source is a vehicle engine and including determining a number of rotations per minute of the engine and delaying the reference signal responsive to the number of rotations per minute.

11. The method of claim 10, including shifting the phase angle of the reference signal.

12. The method of claim 8, including determining how much to delay the reference signal using a lookup table containing a plurality of delay times that correspond to predetermined delay times associated with a plurality of operation conditions of the source of noise.

13. The method of claim 8, wherein the assembly includes a microphone that provides noise information from the vicinity of the actuator, and including:
controlling the actuator using information from the microphone regarding the noise; and
delaying the reference signal to accommodate signal processing delays associated with receiving information from the microphone and communicating signals to the actuator.
